(19) United States
(12) Patent Application Publication
(10) Pub. No.: US 2014/0224257 A1
(43) Pub. Date: Aug. 14, 2014

(54) ADJUSTABLE TELESCOPIC HINGE SYSTEM FOR RELATIVE POSITIONING OF THE UPPER AND LOWER JAW

(71) Applicant: Mark ABRAMSON, Redwood City, CA (US)

(72) Inventor: Mark ABRAMSON, Redwood City, CA (US)

(73) Assignee: Mark ABRAMSON, Redwood City, CA (US)

(21) Appl. No.: 14/175,753

(22) Filed: Feb. 7, 2014

Related U.S. Application Data
(60) Provisional application No. 61/763,813, filed on Feb. 12, 2013.

(51) Int. Cl. A61F 5/56

(52) U.S. Cl. CPC A61F 5/566
USPC 128/848

(57) ABSTRACT

In an embodiment a device adapted to limit relative positioning of a mandible and a maxilla of a user includes a housing and a telescoping member movable within the housing to change a length of the device. An attachment structure extending from the housing pivotably connects the housing to one of the mandible and the maxilla. An attachment structure extending from the telescoping member pivotably connects the telescoping member to the other of the mandible and the maxilla. A stop adjustably positionable in the housing selectively limits movement of the telescoping member within the housing.

### PRIORITY CLAIM

This application claims the benefit of U.S. Provisional Application No. 61/763,813, filed Feb. 12, 2013, the contents of which are incorporated herein by reference.

### TECHNICAL FIELD

This invention relates to device for affecting air flow through the nasal and posterior pharyngeal regions of the upper airway.

### BACKGROUND

Untreated sleep apnea can result in dangerous daytime sleepiness, high blood pressure, heart attack, and stroke. For moderate to severe sleep apnea, a common treatment is the use of a continuous positive airway pressure (CPAP) or an automatic positive airway pressure (APAP) device which ‘splints’ the patient's airway open during sleep by means of a flow of pressurized air into the throat. A patient typically wears a plastic facial mask, which is connected by a flexible tube to a small bedside CPAP machine that generates the required air pressure to keep the patient's airways open during sleep. In addition, an APAP machine will automatically titrate the air pressure as needed to minimize apneas and hypopneas. Advanced models may warm or humidify the air and monitor the patient's breathing to ensure proper treatment. Although CPAP and APAP therapy is effective in reducing apneas, many patients find the therapy extremely uncomfortable and fail to continue using their CPAP machines on a nightly basis, especially in the long term. Further, it is not clear that CPAP therapy reduces hypertension or cardiovascular events in patients who do not have daytime sleepiness; however, the lack of benefit may be partly due to noncompliance with therapy.

Alternatively, custom-fitted sleep disorder devices are available and designed to help patients sleep soundly and continuously, while reducing the effects of sleep apnea. A mandibular splint or mandibular advancement splint (MAS) is a device worn in the mouth that is used to treat obstructive sleep apnea and snoring by moving the lower jaw forward slightly, which tightens the soft tissue and muscles of the upper airway to prevent obstruction of the airway during sleep. The tightening created by the device also prevents the tissues of the upper airway from vibrating as air passes over them—the most common cause of loud snoring. Such devices are commonly made of plastic or acrylic and can be shaped to conform with the dental impression of a patient. These devices can also be somewhat uncomfortable, although many patients find them less bothersome than CPAP mask treatment. Improperly fitted devices can cause teeth to shift over time, leading to significant dental problems. What is needed are additional options for reducing obstruction in airway paths of patients to treat sleep apnea so that patients can choose a the option that is most comfortable for them and therefore allows them to follow through with treatment on a more consistent basis, thereby improving treatment results.

### SUMMARY

In an embodiment, a device adapted to limit relative positioning of a mandible and a maxilla of a user comprises a housing including an inner channel and a telescoping member movable within the inner channel to change a length of the device. The device further comprises a first attachment structure extending from the housing for pivotably connecting the housing to one of the mandible and the maxilla and a second attachment structure extending from the telescoping member for pivotably connecting the telescoping member to the other of the mandible and the maxilla. The device further comprises a stop adjustably positionable to selectively limit movement of the telescoping member within the inner channel.

In an embodiment, the stop is positionable within the inner channel to block the telescoping member at a minimum length of the device. In an embodiment, the at least a portion of a length of the inner channel is threaded and the stop is a set screw with threads mateable with the threads of the inner channel so that the stop is advanceable along the threads of the inner channel to select the minimum length. In an embodiment, the telescoping member is cylindrical in cross-section and the housing is cylindrical in cross-section.

In an embodiment, a proximal end of the telescoping member extends from the housing and a distal end of the telescoping member is moveable within the inner channel and a cross-section of the distal end of the telescoping member is larger than a cross-section of the proximal end of the telescoping member. The inner channel narrows at a proximal end of the inner channel so as to block movement of the telescoping member when the distal end of the telescoping member contacts the narrowed proximal end of the inner channel.

In an embodiment, the first attachment structure is a fin that extends downward relative to the housing when the device is fixed in a mouth of a user and the second attachment structure is a fin that extends upward relative to the telescoping member when the device is fixed in a mouth of a user. In an embodiment, the first attachment structure and the second attachment structure are configured to be attached to attachment structure points fitted to teeth of the mandible and maxilla, respectively.

In an embodiment, the telescoping member and the housing are fabricated from one or more of a surgical grade metal and a thermoplastic resin.

In an embodiment, a system adapted to limit relative positioning of a mandible and a maxilla of a user comprises a pair of devices connectable between the mandible and the maxilla and positioned on opposite sides of the dental arches of the mandible and the maxilla. Each of the devices further comprises a housing including an inner channel and a telescoping member movable within the inner channel to change a length of the device. Each device further comprises a first attachment structure extending from the housing for pivotably connecting the housing to one of the mandible and the maxilla and a second attachment structure extending from the telescoping member for pivotably connecting the telescoping member to the other of the mandible and the maxilla. Each device further comprises a stop adjustably positionable to selectively limit movement of the telescoping member within the inner channel.

### BRIEF DESCRIPTION OF THE DRAWINGS

Further details of the present invention are explained with the help of the attached drawings in which:

FIG. 1 is a perspective view of a device for restricting relative movement of a maxilla and mandible of a user in a first arrangement, in accordance with an embodiment.

FIG. 2 is a perspective view of the device of FIG. 1 in a second arrangement.

FIG. 3 is an exploded view of the device of FIG. 1.

FIG. 4 is a cross-sectional view of the device of FIG. 1 at a minimum length.

FIG. 5 is a cross-sectional view of the device of FIG. 1 at a maximum length.

FIG. 6 is a cross-sectional view of the device of FIG. 1 at an adjusted minimum length.

FIG. 7 is a cross-sectional view of a device for restricting relative movement of a maxilla and mandible of a user in a first arrangement, in accordance with an alternative embodiment.

FIG. 8 is a perspective view of a device for restricting relative movement of a maxilla and mandible of a user at a maximum length, in accordance with an alternative embodiment.

FIG. 9 is a perspective view of a device for restricting relative movement of a maxilla and mandible of a user at an intermediate length, in accordance with an alternative embodiment.

### DETAILED DESCRIPTION

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the first digit of a reference number identifies the embodiment discussed and the drawing in which the embodiment first appears.

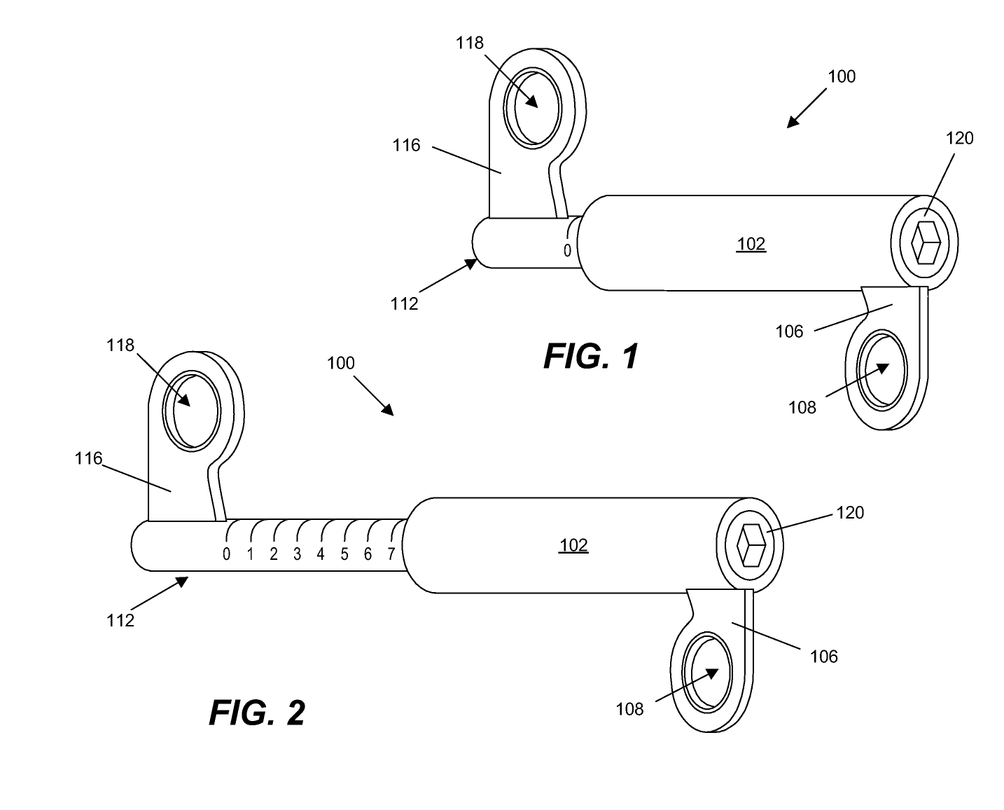

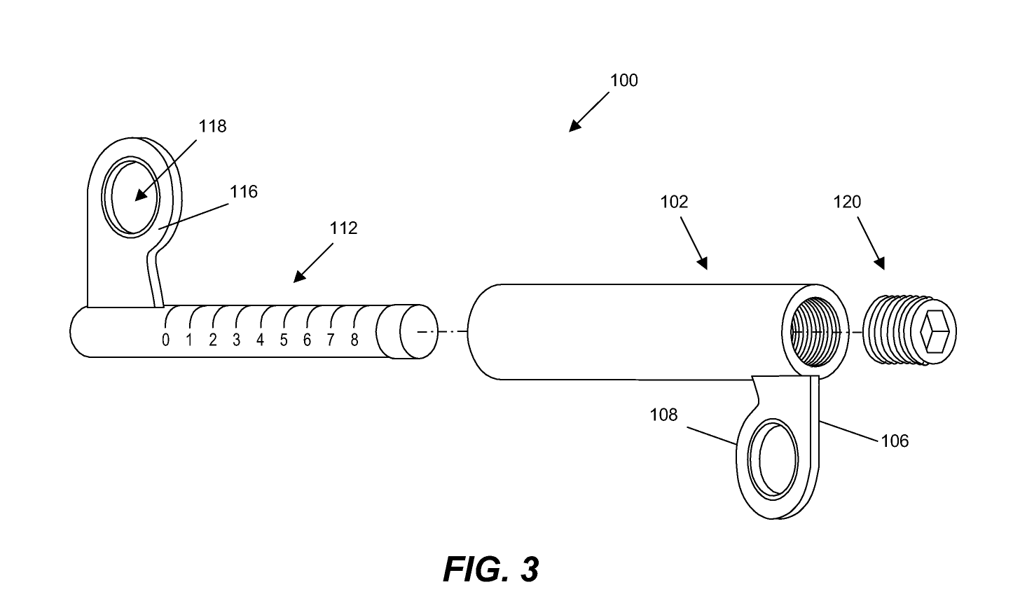

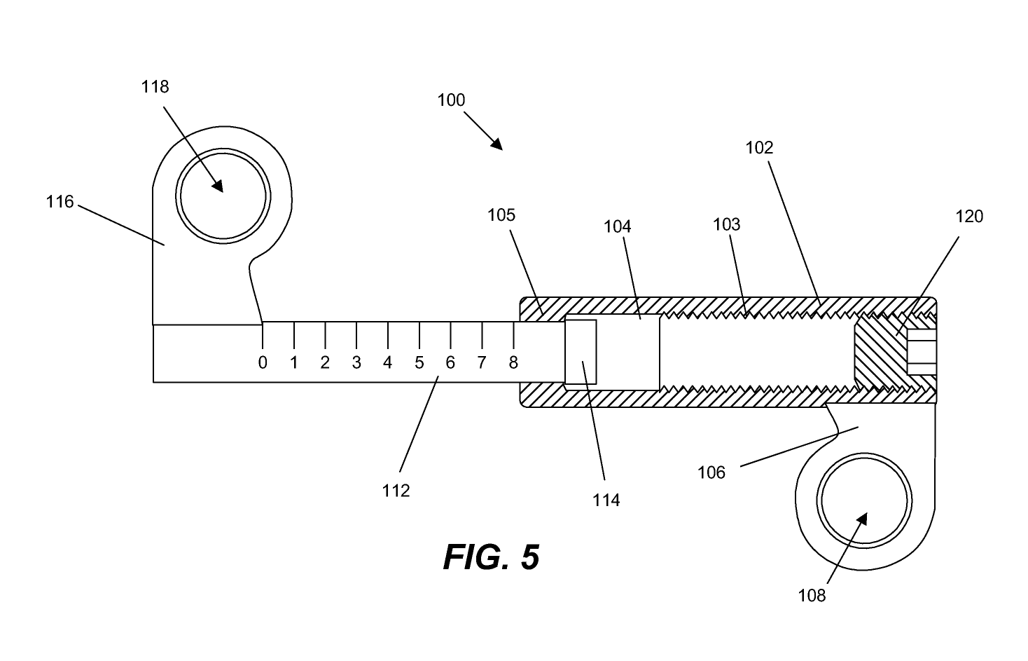

Referring to FIGS. 1-6, a device 100 adapted to limit relative positioning of a mandible and a maxilla of a user in accordance with an embodiment is shown. The device is part of an adjustable, telescoping hinge system and is attachable to the maxillary and mandibular dental arches in position relative to each other. The device 100 is attachable via attachment structures that can be pivotably connected to mounting structures that are themselves connected with the dental arches. Such mounting structures can vary, and can be fixedly attached to the dental arches or removably attached to the dental arches. For example, such mounting structures can include fixed orthodontic wires or a removable mouth guard, preferably though not necessarily plastic, formed to fit securely onto the dental arches and selectively insertable or removable. In an embodiment, the hinge system can comprise two devices positioned on opposite sides of the maxillary and mandibular dental arches.

The device 100 comprises a housing 102 having an inner channel 104. The housing 102 is positioned anterior to a telescoping member 112 of the device 100. As shown, the housing 102 is a tube having a substantially cylindrical cross-sectional shape, although as described below, in other embodiments the housing can have some other cross-sectional shape. A first attachment structure 106 extends from the housing 102 and allows the device 100 to be pivotably connectable to the mandibular dental arch of a user. The first attachment structure 106 is a fin-like extension that can be integrably formed with the housing or separately attached to the housing. The first attachment structure 106 includes a substantially circular eyelet 108 for attachment to the mandibular dental arch via a connector that allows the hinge to pivot smoothly about the connector via rotation. Pivoting occurs with the relative movement of the mandible and maxilla both with posterior and anterior movements of the mandible relative to the maxilla that occur with shifts in the setting of the teeth, and also with the opening and closing of the mouth during eating, speaking, etc.

The inner channel 104 of the housing 102 includes screw threads 103 extending from the anterior end of the housing 102 along at least a portion of the inner channel 104 toward the posterior end of the housing 102. The posterior-most end of the housing 102 has a narrowed (i.e. necked) section 105 comprising an internal cross-section (e.g., an internal diameter, where the housing is a tube) that is narrower than the rest of the inner channel 104. The narrowed section 105 acts as a blocking structure limiting motion of the telescoping member 112 within the inner channel 104, thereby defining a maximum length of the device 100.

As shown, the telescoping member 112 is a rod having a larger (i.e. flared) external diameter anterior end 114 that is arranged in the tube. The telescoping member 112 can move freely through the inner channel 104 of the housing 102 in a posterior direction until contacting the narrowed section 105 having a diameter smaller than the anterior end 114 of the telescoping member. The narrowed section 105 impedes further posterior movement of the telescoping member 112 and prevents the telescoping member 112 from slipping through the posterior end of the housing 102 and separating from the housing 102.

The device 100 further comprises the telescoping member 112. As shown, the telescoping member 112 is the posterior-most component and is a rod that slides telescopingly back and forth through the tube. In other embodiments, the telescoping member 112 can have some other cross-sectional shape to substantially complement the inner channel 104 of the housing 102. In an embodiment, the telescoping member 112 can be arranged within the housing 102 by inserting the telescoping member 112 into the anterior end of the inner channel 104 of the housing 102 and urging the telescope member 112 through the posterior end of the inner channel 104, positioning the telescoping member 112 so that it extends out of the posterior end of the housing 102. A second attachment structure 116 can then be connected with the telescoping member 112 positioned within the inner channel 104. The second attachment structure 116 extends from the telescoping member 112 and allows the device 100 to be pivotably connectable to the maxillary dental arch. The second attachment structure 116 is a fin-like extension that includes a substantially circular eyelet 118 for attachment to the maxillary dental arch via a connector (not shown) that allows the hinge to pivot smoothly about the connector via rotation.

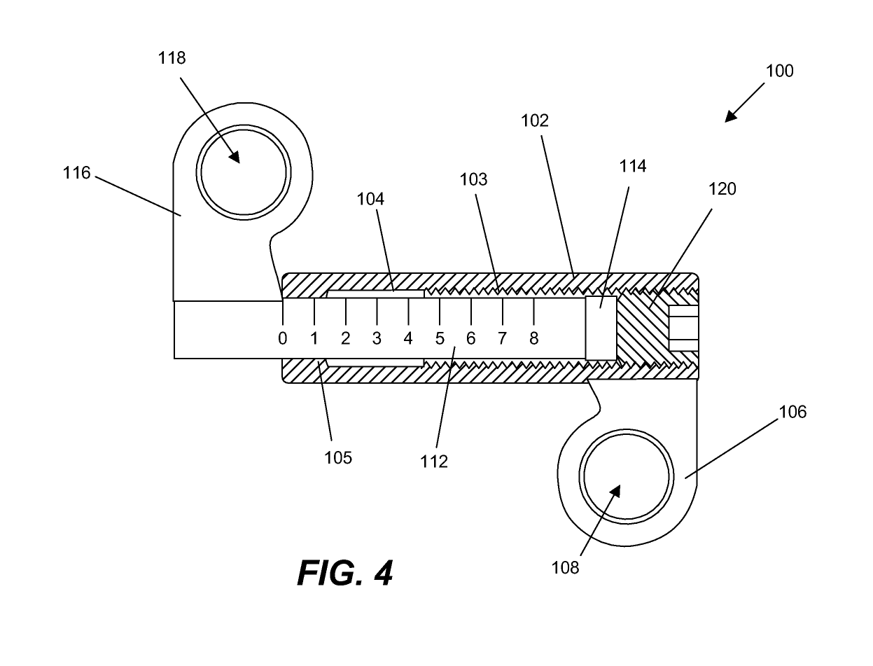

The device 100 further comprises a stop 120 adjustably positionable to selectively limit movement of the telescoping member 112 within the inner channel 104. In an embodiment, the stop 120 can comprise an adjustment screw threaded into the anterior end of the tube and advanced along the inner screw thread of the tube as desired. When the rod is moved forward in the tube, the anterior-most end of the rod will hit the adjustment screw (FIG. 4), which creates a stop that holds the lower mandibular dental arch segment from moving in a posterior direction relative to the upper maxillary dental arch segment but allows forward or anterior movement of the lower mandibular dental arch by means of the rod's ability to slide in a posterior direction in the tube. As shown, the adjustment screw has an internal hex receptor for an Allen wrench to turn the screw. When turned to move posterior in the tube it changes the position of the stop making the hinge adjustable and the rod's ability to move freely in the posterior direction makes it telescopic. In other embodiments, some other type of mating feature can be used for advancing and retreating the adjustment screw.

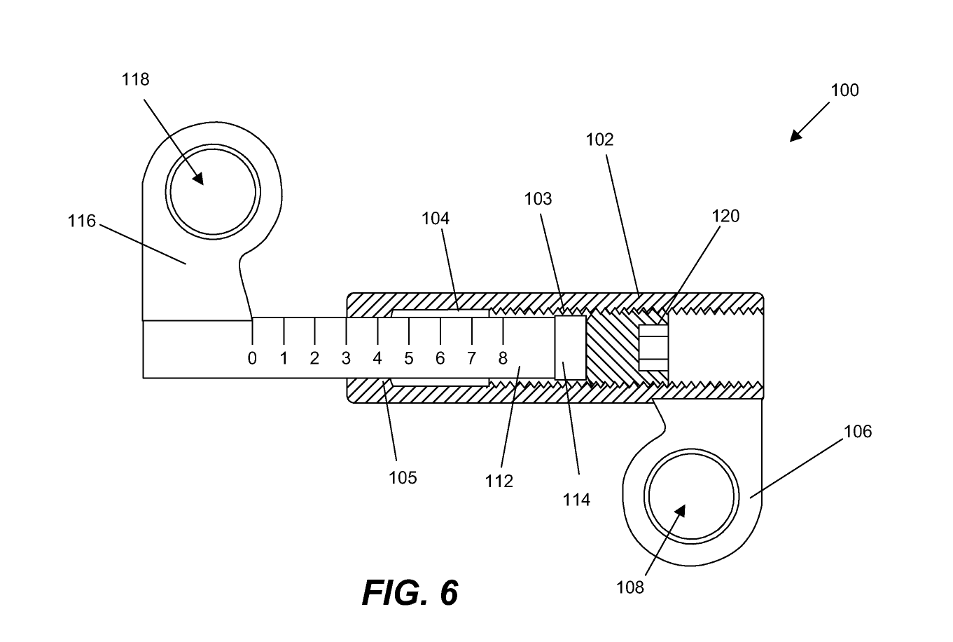

As shown in FIGS. 1-6, the telescoping member 112 can include indicia formed along the length of the telescopic member that can indicate, when the telescoping member is fully seated in the inner channel, how much adjustment has been made to the positioning of the stop. The indicia shown represent millimeters (mm) of distance from the minimum length of the device. As shown in FIG. 6, the stop 120 has been advanced along the threaded portion 103 of the inner channel so that the minimum length of the device 100 is 3 mm larger than the absolute minimum (represented by 0 mm) dictated by the physical obstruction of the attachment structure 116 of the telescoping member 112 with the housing 102.

The housing, telescoping member, attachment structures and stop can each comprise a metal, or some other suitable material, for example a thermoplastic resin such as polyetheretherketone (PEEK), and the like, or a combination of materials. As mentioned above, the housing and the first attachment structure can be integrally manufactured or separately manufactured and subsequently attached. Further, the housing and the first attachment structure can comprise the same or different materials, or combinations of materials. Further, in alternative embodiments, the telescoping member and the second attachment structure can be integrally manufactured or separately manufactured and subsequently attached. Further, the telescoping member and the second attachment structure can comprise the same or different materials, or combinations of materials. Selection of materials can be based on myriad different concerns, including whether the device is to be attached to fixed orthodontic wires or a removable, soft material. A mouth guard can assist in reducing discomfort from harder, and/or potentially more abrasive materials, for example. Other concerns include device cost, durability, practicality, machinability, etc.

In other embodiments, the telescoping member can be the posterior-component connected with the mandibular dental arch and the housing can be the anterior-component connected with the maxillary dental arch. In other embodiments, the telescoping member can be the anterior-component connected with the maxillary dental arch and the housing can be the posterior-component connected with the mandibular dental arch. In other embodiments, the telescoping member can be the anterior-component connected with the mandibular dental arch and the housing can be the posterior-component connected with the maxillary dental arch.

In accordance with some embodiments of systems, a single device comprising an adjustable telescoping hinge or a pair of devices comprising adjustable telescoping hinges positioned on respective sides of the jaws can be used. Further, in embodiments the housing of each device can include a venting port to prevent air from dampening movement of the telescoping member through the housing, or alternatively, the housing can include some material, such as a compressible gas, to purposely and controllably dampen posterior movement of the lower jaw relative to the upper jaw.

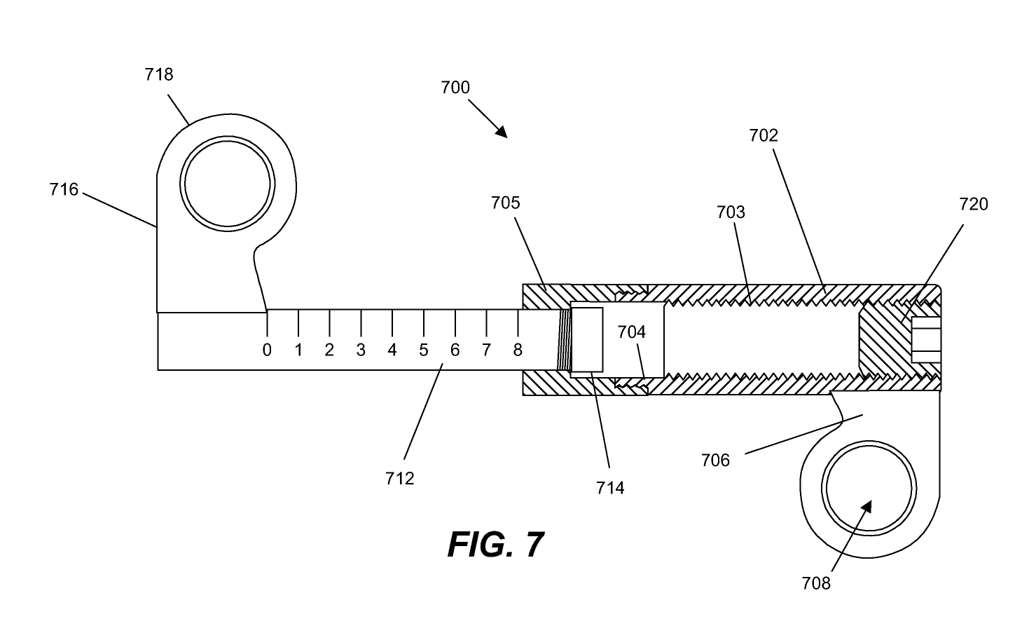

Referring to FIG. 7, in alternative embodiments, some other technique(s) and/or structures can be used for positioning the telescoping member 712 inside of the inner channel 704. For example, the narrowed end 705 of the inner channel 704 can be separately threaded onto the rest of the inner channel 704 after a main length of a telescoping member 712 is positioned through the narrowed end 705 and a threaded anterior end 714 of the telescoping member is separately mated with the main length of telescoping member 712 and advanced until the threaded anterior end 714 is fully seated onto the main length of the telescoping member 712. Separating the telescoping member into two threaded pieces 712, 714 and separating the inner channel into two pieces 704, 705 can simplify manufacturing and/or allow the attachment structure 716 of the telescoping member to be integrally formed.

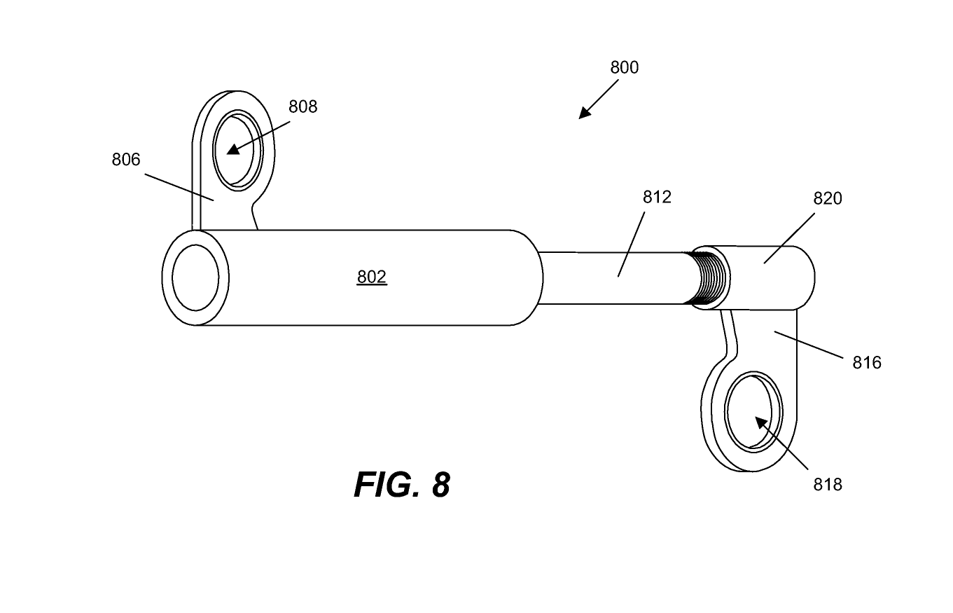

In alternative embodiments, some other technique(s) and/or structures can be used to adjust the minimum length of the device. FIG. 8 illustrates a device 800 adapted to limit relative positioning of a mandible and a maxilla of a user in accordance with an alternative embodiment. As with the previously described embodiment, the device 800 comprises a housing 802 (e.g., a tube), a first attachment structure 806 (e.g., a fin-like extension), and a telescoping member 812 (e.g., a rod) that can telescope within the tube. However, the telescoping member is threadably mated with a threaded stop 820 that is advanced and retreated along the outer surface of the telescoping member to limit the minimum length of the device. The absolute minimum of the device 800 is determined by the maximum distance the stop 820 can be advanced along the rod, which can be defined by the distance along the rod that is threaded. In such an embodiment, an attachment structure 818 (e.g., a fin-like extension) that extends from the stop 820.

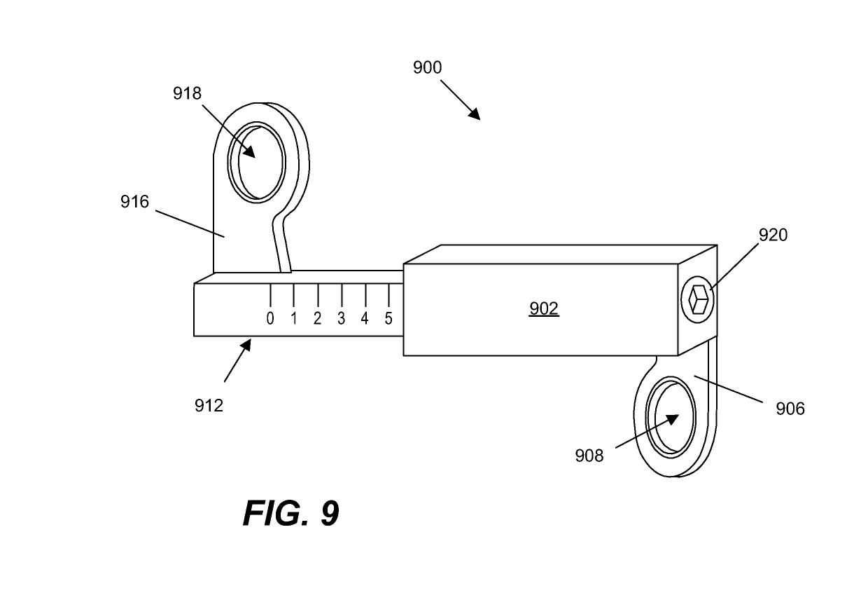

In alternative embodiments, some other technique(s) and/or structures need not be tubular or cylindrical in shape. FIG. 9 illustrates a device 900 adapted to limit relative positioning of a mandible and a maxilla of a user in accordance with an alternative embodiment. As with the previously described embodiment, the device 900 comprises a housing 902, a first attachment structure 906 (e.g., a fin-like extension), a telescoping member 912 that can telescope within the housing, and a second attachment structure 916 (e.g., a fin-like extension). However, the housing 902 has a cross-sectional shape that is not circular, but rather rectangular in shape having a rectangular inner channel. The telescoping member 912 has a cross-sectional shape that complements the rectangular inner chamber of the housing 902. A set screw 920 can be used to push a rectangular stop into the inner chamber to adjust a minimum length of the device 900. The embodiment of FIG. 9 is provided to illustrate the variety of different shapes with which devices in accordance with embodiment can take, and is intended to expand the scope of the embodiments rather than limit the scope of the embodiments. In practice, a device with sharp edges may not be comfortable or practical for use in the mouth of a patient unless encapsulated with a soft outer layer. However, other shapes, such as ovoid shapes may be suitable for use in the mouth of a patient without protective coatings.

The foregoing description of the present invention has been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

1. A device adapted to limit relative positioning of a mandible and a maxilla of a user, comprising:
a housing including an inner channel;
a first attachment structure extending from the housing for pivotably connecting the housing to one of the mandible and the maxilla;
a telescoping member movable within the inner channel to change a length of the device;
a second attachment structure extending from the telescoping member for pivotably connecting the telescoping member to the other of the mandible and the maxilla; and
a stop adjustably positionable to selectively limit movement of the telescoping member within the inner channel.

2. The device of claim 1, wherein the stop is positionable within the inner channel to block the telescoping member at a minimum length of the device.

3. The device of claim 2, wherein at least a portion of a length of the inner channel is threaded and the stop is a set screw with threads mateable with the threads of the inner channel so that the stop is advanceable along the threads of the inner channel to select the minimum length.

4. The device of claim 1,
wherein a proximal end of the telescoping member extends from the housing and a distal end of the telescoping member is moveable within the inner channel;
wherein a cross-section of the distal end of the telescoping member is larger than a cross-section of the proximal end of the telescoping member; and
wherein the inner channel narrows at a proximal end of the inner channel so as to block movement of the telescoping member when the distal end of the telescoping member contacts the narrowed proximal end of the inner channel.

5. The device of claim 1, wherein the first attachment structure is a fin that extends downward relative to the housing when the device is fixed in a mouth of a user.

6. The device of claim 1, wherein the second attachment structure is a fin that extends upward relative to the telescoping member when the device is fixed in a mouth of a user.

7. The device of claim 1, wherein the telescoping member is cylindrical in cross-section and the housing is cylindrical in cross-section.

8. The device of claim 1, wherein the telescoping member and the housing are fabricated from one or more of a surgical grade metal and a thermoplastic resin.

9. The device of claim 1, wherein the first attachment structure and the second attachment structure are configured to be attached to attachment structure points fitted to teeth of the mandible and maxilla, respectively.

10. A system adapted to limit relative positioning of a mandible and a maxilla of a user, comprising:
a first device including
a housing having an inner channel,
a housing attachment structure extending from the housing for pivotably connecting the housing to one of the mandible and the maxilla,
a telescoping member movable within the inner channel to change a length of the device, and
a telescoping member attachment structure extending from the telescoping member for pivotably connecting the telescoping member to the other of the mandible and the maxilla, and
a stop adjustably positionable to selectively limit movement of the telescoping member within the inner channel; and
a second device including
a housing having an inner channel,
a housing attachment structure extending from the housing for pivotably connecting the housing to one of the mandible and the maxilla,
a telescoping member movable within the inner channel to change a length of the device, and
a telescoping member attachment structure extending from the telescoping member for pivotably connecting the telescoping member to the other of the mandible and the maxilla, and
a stop adjustably positionable to selectively limit movement of the telescoping member within the inner channel;
wherein the first device and the second device are positionable on opposite sides of the mandible and the maxilla.

11. The system of claim 10, wherein for each of the first device and the second device, the stop is positionable within the inner channel to block the telescoping member at a minimum length of the respective device.

12. The system of claim 11, wherein for each of the first device and the second device, at least a portion of a length of the inner channel is threaded and the stop is a set screw with threads mateable with the threads of the inner channel so that the stop is advanceable along the threads of the inner channel to select the minimum length.

13. The system of claim 10, wherein for each of the first device and the second device
a proximal end of the telescoping member extends from the housing and a distal end of the telescoping member is moveable within the inner channel,
a cross-section of the distal end of the telescoping member is larger than a cross-section of the proximal end of the telescoping member, and
the inner channel narrows at a proximal end of the inner channel so as to block movement of the telescoping member when the distal end of the telescoping member contacts the narrowed proximal end of the inner channel.

14. The system of claim 10, wherein for each of the first device and the second device, the first attachment structure is a fin that extends downward relative to the housing when the device is fixed in a mouth of a user.

15. The system of claim 10, wherein for each of the first device and the second device, the second attachment structure is a fin that extends upward relative to the telescoping member when the device is fixed in a mouth of a user.

16. The system of claim 10, wherein t for each of the first device and the second device, the telescoping member is cylindrical in cross-section and the housing is cylindrical in cross-section.

17. The system of claim 10, wherein for each of the first device and the second device, the telescoping member and the housing are fabricated from one or more of a surgical grade metal and a thermoplastic resin.

18. The system of claim 10, wherein for each of the first device and the second device, the first attachment structure and the second attachment structure are configured to be attached to attachment structure points fitted to teeth of the mandible and maxilla, respectively.

19. A device adapted to limit relative positioning of a mandible and a maxilla of a user, comprising:
a housing including an inner channel, wherein at least a portion of a length of the inner channel is threaded;
a first attachment structure extending from the housing for pivotably connecting the housing to one of the mandible and the maxilla;
a telescoping member movable within the inner channel to change a length of the device;
a second attachment structure extending from the telescoping member for pivotably connecting the telescoping member to the other of the mandible and the maxilla; and
a set screw with threads mateable with the threads of the inner channel so that the set crew is advanceable along the inner channel to selectively limit movement of the telescoping member within the inner channel by blocking the telescoping member.

20. The device of claim 19,
wherein the telescoping member is cylindrical in cross-section and the inner channel is cylindrical in cross-section;
wherein a proximal end of the telescoping member extends from the housing and a distal end of the telescoping member is moveable within the inner channel;
wherein a diameter of the distal end of the telescoping member is larger than a diameter of the proximal end of the telescoping member; and
wherein a diameter of the inner channel narrows at a proximal end of the inner channel so as to block movement of the telescoping member when the distal end of the telescoping member contacts the narrowed proximal end of the inner channel.
